(19) United States
(12) Patent Application Publication
(10) Pub. No.: US 2022/0104983 A1
(43) Pub. Date: Apr. 7, 2022

(54) NEGATIVE PRESSURE ROOM WITH SAFETY MANAGEMENT SYSTEM

(71) Applicant: BA ENERGY CO., LTD., Gwangju (KR)

(72) Inventors: Tae Young KANG, Gyeonggi-do (KR); Tae Jun PARK, Gwangju (KR)

(21) Appl. No.: 17/117,531

(22) Filed: Dec. 10, 2020

(30) Foreign Application Priority Data
Oct. 6, 2020 (KR) 10-2020-0128576

(51) Int. Cl. A61G 10/02; F24F 3/167; A61L 9/14; F24F 11/00

(52) U.S. Cl. CPC A61G 10/023; F24F 3/167; A61L 9/14; F24F 2110/40; A61L 2209/134; A61L 2209/14; F24F 11/0001

(57) ABSTRACT

A negative pressure room with a safety management system according to an embodiment of the present disclosure includes an interior space being under negative pressure and is provided in a movable form, so that the negative pressure room can be constructed promptly and economically in the event of a spreading infection, thereby enabling rapid and efficient quarantine treatment and observation of patients with confirmed and suspected infections, the negative pressure room is isolated from a general ward, thereby preventing the possibility of further infection to other patients or medical staff, and real-time monitoring whether negative pressure is appropriately generated in the interior space, air quality in the interior space, and the state of an isolated patient is enabled.

### CROSS-REFERENCE TO RELATED APPLICATION AND CLAIM OF PRIORITY

This application claims priority to Korean Patent Applications No. 10-2020-0128576 filed on Oct. 6, 2020 in the Korean Intellectual Property Office (KIPO), the entire disclosure of which is incorporated by reference herein.

### TECHNICAL FIELD

The present disclosure relates generally to a negative pressure room. More particularly, the present disclosure relates to a negative pressure room with a safety management system, wherein i) the negative pressure room has an interior space being under negative pressure and is provided in a movable form, so that the negative pressure room can be constructed promptly and economically in the event of a spreading infection, thereby enabling rapid and efficient quarantine treatment and observation of patients with confirmed and suspected infections, ii) the negative pressure room is isolated from a general ward, thereby preventing the possibility of further infection to other patients or medical staff, and iii) real-time monitoring whether negative pressure is appropriately generated in the interior space, air quality in the interior space, and the state of an isolated patient is enabled.

### BACKGROUND ART

In general, a negative pressure isolation room is a room that accommodates an infectious patient by isolating the patient from others during treatment to prevent the spread of infectious diseases. In this room, air pressure inside the room is kept lower than atmospheric pressure so that air inside the room or pathogens contained in aerosols or droplets do not escape from the room. The air inside the isolation room is exhausted outside through an exhaust facility with a high-efficiency particulate air (HEPA) filter to remove pathogens from the air.

Negative pressure isolation rooms are used for the purpose of preventing spreading infection of novel infectious diseases, such as Middle East Respiratory Syndrome (MERS), Severe Acute Respiratory Syndrome (SARS), Avian Influenza Human Infection (AIHI), Novel swine-origin influenza A (H1N1), and Ebola hemorrhagic fever (EHF).

However, a typical related-art negative pressure isolation room in a building requires enormous cost and time for installation and thus is seriously problematic in that it is difficult to quickly respond to the spread of novel infectious diseases.

The related-art negative pressure isolation room is further problematic in that there is a risk of further spreading infection within the same ward if there is a problem in the management of medical staff, and in that although the principle of one room for one patient should be followed to treat patients with such novel infectious diseases, the room is often shared by two to six patients due to the lack of negative pressure isolation rooms and cost problems.

Meanwhile, Korean Utility Model Registration No. 20-0406536 (registered on Jan. 12, 2006) proposed a bio-clean room bacterial contamination prevention system having an air pressure control device. The system includes an air pressure control device that has a differential pressure sensor and controls air differential pressure inside an isolation room and a controller that controls the differential pressure device, and is used in an isolation room for isolating patients with infectious diseases, the isolation room being provided with an inlet pipe for allowing inflow of air and an outlet pipe. However, the proposed is only related to the configuration inside an outlet pipe of a negative pressure isolation room in a ward, and has no relation to a mobile negative pressure isolation room.

In addition, filters installed in air pressure control devices used in negative pressure isolation rooms are required to be replaced when their service life is over after long-term use, in which case there is a problem in that viruses attached to the filters may escape outside during the replacement.

### SUMMARY

Accordingly, the present disclosure has been made keeping in mind the above problems occurring in the related art, and an objective of the present disclosure is to provide a negative pressure room with a safety management system, wherein i) the negative pressure room has an interior space being under negative pressure and is provided in a movable form, so that the negative pressure room can be constructed promptly and economically in the event of a spreading infection, thereby enabling rapid and efficient quarantine treatment and observation of patients with confirmed and suspected infections, ii) the negative pressure room is isolated from a general ward, thereby preventing the possibility of further infection to other patients or medical staff, and iii) transfer of an infected patient is enabled while the patient is isolated in the negative pressure room using a trailer, thereby fundamentally preventing the risk of further infection due to patient transfer.

Another aspect of the present disclosure is to provide a negative pressure room with a safety management system, the negative pressure room being capable of enabling real-time monitoring whether negative pressure is appropriately generated in an interior space, air quality in the interior space, and the state of an isolated patient.

Still another aspect of the present disclosure is to provide a negative pressure room with a safety management system, wherein viruses attached to a filter are removed with a chemical sprayed through a chemical sprayer, so that the viruses attached to the filter are prevented from escaping outside when a filter installed in a negative pressure device is replaced after long-term use.

Aspects of the present disclosure are not limited to the aspects mentioned above, and other aspects not mentioned will be clearly understood by those skilled in the art from the following description.

In order to achieve the above aspects, according to one aspect of the present disclosure, there is provided a negative pressure room with a safety management system, the negative pressure room including: a negative pressure room body including an ante-room provided with a first door connected to outside, a patient room provided with a second door connected to the ante-room, and a bathroom provided with a third door connected to the patient room; a negative pressure device installed at the negative pressure room body, and configured to generate negative pressure in an interior space of the negative pressure room body; an air conditioning system installed at the negative pressure room body, and configured to control cooling/heating of the interior space; at least one differential pressure sensor installed at the negative pressure room body; and a display configured to display differential pressure data between the respective rooms measured by the differential pressure sensor in real time.

In a preferred embodiment, the negative pressure room may further include: a body temperature sensor and a pulse sensor attached to an isolated patient located in the interior space of the negative pressure room body; and a temperature sensor, a humidity sensor, a fine dust sensor, a vibration sensor, or a fire detection sensor installed at a predetermined position of the negative pressure room body, wherein the display may display data measured by these sensors in real time.

In a preferred embodiment, the negative pressure may be generated such that an absolute value thereof is high in the order of the bathroom, the patient room, and the ante-room.

In a preferred embodiment, the negative pressure device may be installed in each of the ante-room and the patient room, and a variable communication port and a blowing fan may be further provided, the variable communication port being installed in a wall between the bathroom and the patient room and configured to allow communication between the bathroom and the patient room or release communication between the bathroom and the patient room, the blowing fan being installed in the variable communication port, wherein negative pressure of the bathroom may be generated by the variable communication port and the blowing fan.

In a preferred embodiment, the negative pressure device may include: a body; a motor installed inside or outside the body; an intake part formed at a side of the body, and configured to allow drawing of air in the interior space therethrough; a filter part provided inside the body, and configured to purify air drawn through the intake part; and an exhaust part formed on the outside of the body, and configured to allow exhaust of air purified by the filter part therethrough.

In a preferred embodiment, the filter part may include a plurality of filters.

In a preferred embodiment, the filter part may include: a casing in which a filter is accommodated; an air sprayer installed at the casing, and configured to generate an air curtain in a vertical direction; and at least one chemical sprayer installed at the casing at a position between the air curtain and the filter, and configured to spray a chemical toward the filter, wherein the air curtain may prevent the viruses attached to the filter from escaping back in the form of an aerosol due to the chemical sprayer.

In a preferred embodiment, the chemical sprayer may spray an aqueous sodium hypochlorite solution.

In a preferred embodiment, the first door and the second door may be controlled so as not to be opened simultaneously.

In a preferred embodiment, the negative pressure room may further include a sterilization system installed at a position in the vicinity of the first door of the ante-room, and configured to spray a chemical onto a person entering and leaving the ante-room.

The present disclosure has the following excellent effects.

According to a negative pressure room with a safety management system according to the present disclosure, i) the negative pressure room has an interior space being under negative pressure and is provided in a movable form, so that it is possible to construct the negative pressure room promptly and economically in the event of a spreading infection, thereby enabling rapid and efficient quarantine treatment and observation of patients with confirmed and suspected infections, ii) the negative pressure room is isolated from a general ward, so that it is possible to prevent the possibility of further infection to other patients or medical staff, and iii) it is possible to transfer an infected patient while the patient is isolated in the negative pressure room using a trailer, thereby fundamentally preventing the risk of further infection due to patient transfer.

In addition, according to the negative pressure room with the safety management system, it is possible to implement data, which is measured by various sensors, on whether negative pressure is appropriately generated in the interior space, air quality in the interior space, and the state of an isolated patient in real time as a web or application based on Internet of Things (IoT), thereby enabling real-time monitoring by the isolated patient or an administrator.

In addition, according to the negative pressure room with the safety management system, viruses attached to a filter are removed with a chemical sprayed through a chemical sprayer, so that it is possible to prevent the viruses attached to the filter from escaping outside when a filter is replaced after long-term use, and during the spraying of the chemical, it is possible to prevent the viruses attached to the filter from escaping in the form of an aerosol by the air curtain generated in the vertical direction.

### BRIEF DESCRIPTION OF THE DRAWINGS

FIG. 1 is a photograph illustrating a negative pressure room with a safety management system according to the present disclosure.

FIG. 2 is a sectional view illustrating the negative pressure room with the safety management system according to the present disclosure.

FIG. 3 is a view illustrating a negative pressure device in the negative pressure room with the safety management system according to the present disclosure.

FIG. 4 is a view illustrating a filter part of the negative pressure device in the negative pressure room with the safety management system according to the present disclosure.

### DETAILED DESCRIPTION

The terminology which is used in common will be used for the purpose of description and not of limitation. Furthermore, terms and words used by the applicant may be used for special cases. In this case, the meaning of terms or words must be understood with due regard to the meaning expressed in the specification rather than taking into account only the basic meaning of the terms and words.

Hereinafter, the technical configuration of the present disclosure will be described in detail with reference to an exemplary embodiment illustrated in the accompanying drawings.

The present disclosure may, however, be embodied in different forms and should not be construed as limited to the embodiment set forth herein. Throughout the drawings, the same reference numerals will refer to the same or like parts.

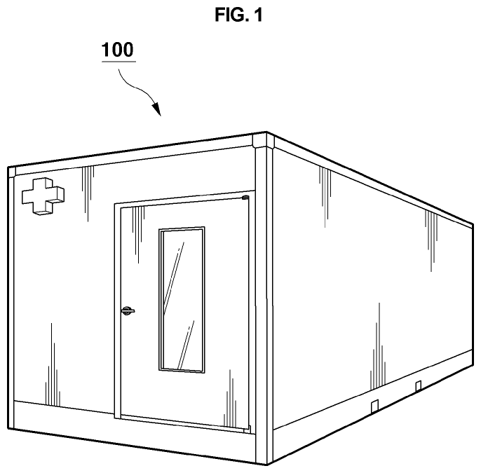

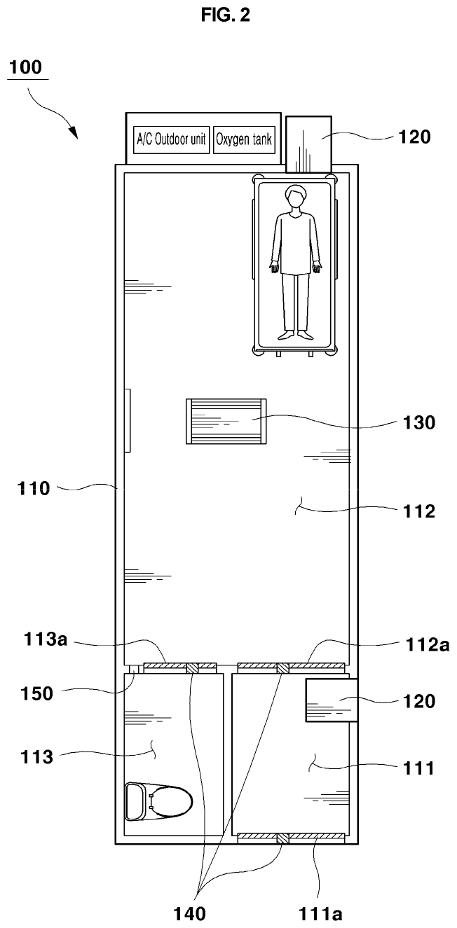

FIG. 1 is a photograph illustrating a negative pressure room with a safety management system according to the present disclosure, and FIG. 2 is a sectional view illustrating the negative pressure room with the safety management system according to the present disclosure.

Referring to FIGS. 1 to 2, the negative pressure room 100 with the safety management system according to the embodiment of the present disclosure is a negative pressure room having an interior space that is under negative pressure, provided in a movable form, and capable of enabling real-time monitoring whether negative pressure is appropriately generated in the interior space, air quality in the interior space, and the state of an isolated patient. The negative pressure room 100 includes a negative pressure room body 110, a negative pressure device 120, an air conditioning system 130, a differential pressure sensor 140, and a display.

The negative pressure room body 110 has the interior space divided into an ante-room 111, a patient room 112, and a bathroom 113.

Here, the ante-room 111 is provided with a first door 111a connected to the outside, the patient room 112 is provided with a second door 112a connected to the ante-room 111, and the bathroom 113 is provided with a third door 113a connected to the patient room 112.

The negative pressure device 120 is a means for generating negative pressure in the interior space, and is installed in the negative pressure room body 110.

For example, the negative pressure device 120 may be provided in each of the ante-room 111, the patient room 112, and the bathroom 113.

As another example, the negative pressure device 120 may not be provided in the bathroom 113, but may be provided only in each of the ante-room 111 and the patient room 112.

In this case, a variable communication port 150 is installed in a wall between the bathroom 113 and the patient room 112.

At this time, the variable communication port 150 serves to allow communication between the bathroom 113 and the patient room 112, or to release communication between the bathroom 113 and the patient room 112. In the variable communication port 150, a blowing fan may be further installed.

Thus, the bathroom 113 communicates with the patient room 112 by the variable communication port 150 and the blowing fan without requiring the provision of a separate negative pressure device, so that negative pressure is generated in an interior space of the bathroom 113.

In addition, by the negative pressure device 120, an absolute value of negative pressure is maintained high in the order of the bathroom 113, the patient room 112, and the ante-room 111, and preferably, a difference in negative pressure between the bathroom 113 and the patient room 112 is maintained at least −2.5 pa, and a difference in negative pressure between the patient room 112 and the ante-room 111 is maintained at least −2.5 pa.

This ensures that even when the first door 111a is opened, external air is allowed into the interior space, but internal air does not escape from the space, and when the second door 112b is opened, contaminated air inside the patient room 112 does not escape into the ante-room 111.

In addition, the first door 111a and the second door 112a may be controlled so as not to be opened simultaneously.

This is to fundamentally prevent internal and external pressures from becoming equal by preventing simultaneous opening of the first door 111a and the second door 112a.

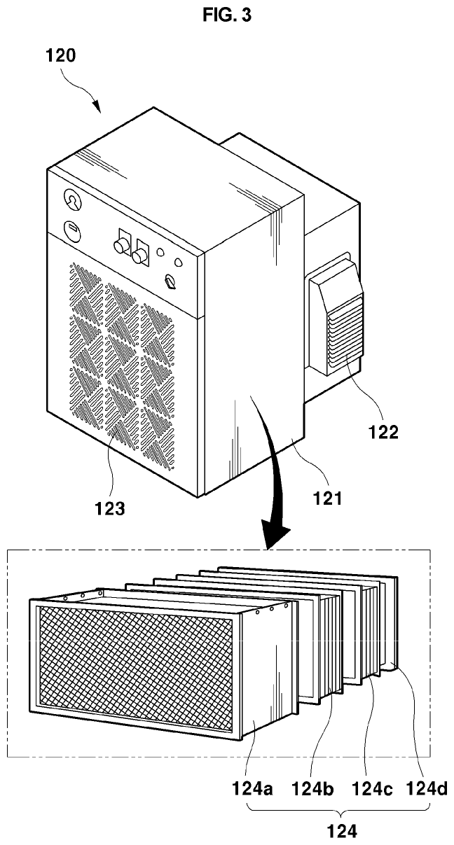

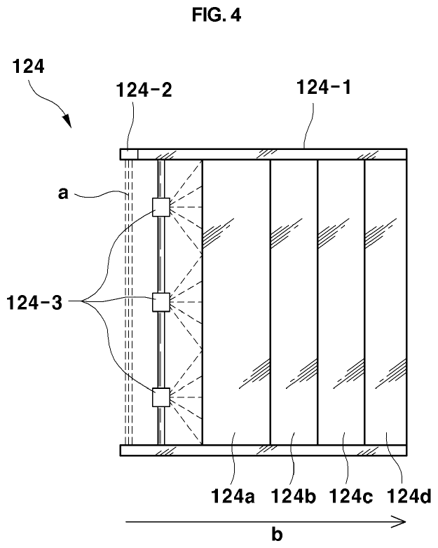

FIG. 3 is a view illustrating a negative pressure device in the negative pressure room with the safety management system according to the present disclosure, and FIG. 4 is a view illustrating a filter part of the negative pressure device.

Hereinafter, the negative pressure device 120 will be described in more detail with reference to FIGS. 3 and 4.

Referring to FIGS. 3 and 4, the negative pressure device 120 may include a body 121, a motor 122, an intake part 123, the filter part 124, and an exhaust part.

Here, the body 121 is provided with an on/off switch for controlling the negative pressure device 120, a negative pressure value setting panel, etc. The motor 122 is installed inside or outside the body 121. The intake part 123 is formed on a side of the body 121 and serves to allow drawing of air in the interior space therethrough. The filter part 124 is provided inside the body 121 and serves to purify air drawn through the intake part 123. The exhaust part is formed on an outside of the body 121 and serves to allow exhaust of air purified by the filter part 124 therethrough.

In addition, the filter part 124 may include a plurality of filters.

For example, the filter part 124 may include four filters 124a, 124b, 124c, and 124d. In this case, the first filter 124a may function by the principle of filtering out contaminants caught between filter fibers. The second filter 124b may function by principle of filtering out contaminants rushing to filter fibers along a streamline flow without escaping from the filter fibers. The third filter 124c may function by the principle of filtering out contaminants caught in the blocking radius of filter fibers. The fourth filter 124d may function by the principle of increasing the blocking effect due to collisions between particles.

In addition, the filter part 124 may include a casing 124-1, an air sprayer 124-2, and a chemical sprayer 124-3.

Here, the filters 124a, 124b, 124c, and 124d are accommodated in the casing 124-1.

In addition, the air sprayer 124-2 is installed at the casing 124-1, and serves to generate an air curtain a in the vertical direction.

In addition, at least one chemical sprayer 124-3 is installed at the casing 124-1 at a position between the air curtain a and the filters 124a, 124b, 124c, and 124d, and serves to spray a chemical toward the filters 124a, 124b, 124c, and 124d to remove contaminants such as viruses attached to the filters 124a, 124b, 124c, and 124d.

In addition, the chemical is not limited as long as it is a chemical in the form of an aqueous solution capable of removing viruses, but is preferably an aqueous sodium hypochlorite solution.

At this time, the air curtain a serves to prevent the viruses attached to the filters 124a, 124b, 124c, and 124d from escaping back in the form of an aerosol due to the chemical sprayer 124-3.

In other words, with the air curtain a generated in the vertical direction, when the chemical is sprayed in an air intake direction b through the chemical sprayer 124-3, the viruses attached to the filters 124a, 124b, 124c, and 124d are prevented from escaping to outside of the casing 124-1 (in the reverse direction of the air intake direction b).

The air conditioning system 130 is installed at the negative pressure room body 110, and serves to control cooling/heating of the interior space.

For example, the air conditioning system 130 may be installed in the patient room 112 in a form attached to the ceiling.

At least one differential pressure sensor 140 is installed at the negative pressure room body 110, and serves to measure a difference in negative pressure between the respective rooms.

For example, three differential pressure sensors 140 may be respectively installed at a position of or in the vicinity of the first door 111a, a position of or in the vicinity of the second door 112a, and a position of or in the vicinity of the third door 113a, so that a difference in negative pressure between neighboring rooms may be measured.

In addition, the differential pressure sensor 140 may detect a difference in negative pressure between the respective rooms, and when the difference in negative pressure is less than 2.5 pa, may generate an alarm.

The display serves to display differential pressure data between the respective rooms measured by the differential pressure sensors 140 in real time, so that whether the differential pressure between the rooms is appropriate is monitored in real time.

In addition, the negative pressure room 100 with the safety management system according to the present disclosure may further include: a body temperature sensor and a pulse sensor attached to the isolated patient located in the interior space of the negative pressure room body 110; and a temperature sensor, a humidity sensor, a fine dust sensor, a vibration sensor, or a fire detection sensor installed at a predetermined position of the negative pressure room body 110.

In this case, the display displays data measured by these sensors in real time.

In other words, in the negative pressure room 100 with the safety management system according to the present disclosure, by implementing data, which is measured by various sensors, on whether negative pressure is appropriately generated in the interior space, air quality in the interior space, and the state of the isolated patient in real time as a web or application based on Internet of Things (IoT), there is an advantage of enabling real-time monitoring by the isolated patient or an administrator.

In addition, the negative pressure room 100 with the safety management system according to the present disclosure may further include a sterilization system provided at a position in the vicinity of the first door 111a of the ante-room 111.

The sterilization system is provided with a plurality of spraying means, and serves to spray a chemical onto a person entering and leaving the ante-room 111 to remove viruses attached to the person and at the same time form a passage section in the vicinity of the ante-room 111 into a sterilization space.

For example, the chemical may be an aqueous sodium hypochlorite solution.

As described above, according to the negative pressure room with the safety management system according to the present disclosure, i) the negative pressure room has the interior space being under negative pressure and is provided in a movable form, so that it is possible to construct the negative pressure room promptly and economically in the event of a spreading infection, thereby enabling rapid and efficient quarantine treatment and observation of patients with confirmed and suspected infections, ii) the negative pressure room is isolated from a general ward, so that it is possible to prevent the possibility of further infection to other patients or medical staff, iii) it is possible to transfer an infected patient while the patient is isolated in the negative pressure room using a trailer, thereby fundamentally preventing the risk of further infection due to patient transfer, and iv) it is possible to implement data, which is measured by various sensors, on whether negative pressure is appropriately generated in the interior space, air quality in the interior space, and the state of the isolated patient in real time as a web or application based on Internet of Things (IoT), thereby enabling real-time monitoring by the isolated patient or an administrator.

Furthermore, viruses attached to the filters are removed with the chemical sprayed through the chemical sprayer, so that it is possible to prevent the viruses attached to the filters from escaping outside when a filter is replaced after long-term use, and during the spraying of the chemical, it is possible to prevent the viruses attached to the filters from escaping in the form of an aerosol by the air curtain generated in the vertical direction.

Although the exemplary embodiment of the present disclosure has been described in detail above, the scope of the present disclosure is not limited to the embodiment. Accordingly, those skilled in the art will appreciate that various substitutions, modifications, and changes are possible, without departing from the spirit and scope of the present disclosure as defined by the accompanying claims.

A mobile negative pressure room according to the present disclosure can find application as a negative pressure isolation room used for preventing spreading infection of novel infectious diseases, such as Middle East Respiratory Syndrome (MERS), Severe Acute Respiratory Syndrome (SARS), Avian Influenza Human Infection (AIHI), Novel swine-origin influenza A (H1N1), Ebola hemorrhagic fever (EHF), and Coronavirus Disease-19 (COVID-19). Furthermore, a plurality of negative pressure rooms can be provided and used as a negative pressure room system.

What is claimed is:

1. A negative pressure room with a safety management system, the negative pressure room comprising:
a negative pressure room body including an ante-room provided with a first door connected to outside, a patient room provided with a second door connected to the ante-room, and a bathroom provided with a third door connected to the patient room;
a negative pressure device installed at the negative pressure room body, and configured to generate negative pressure in an interior space of the negative pressure room body;
an air conditioning system installed at the negative pressure room body, and configured to control cooling/heating of the interior space;
at least one differential pressure sensor installed at the negative pressure room body; and
a display configured to display differential pressure data between the respective rooms measured by the differential pressure sensor in real time.

2. The negative pressure room of claim 1, further comprising:
a body temperature sensor and a pulse sensor attached to an isolated patient located in the interior space of the negative pressure room body; and
a temperature sensor, a humidity sensor, a fine dust sensor, a vibration sensor, or a fire detection sensor installed at a predetermined position of the negative pressure room body,
wherein the display displays data measured by these sensors in real time.

3. The negative pressure room of claim 1, wherein the negative pressure is generated such that an absolute value thereof is high in the order of the bathroom, the patient room, and the ante-room.

4. The negative pressure room of claim 1, wherein the negative pressure device is installed in each of the ante-room and the patient room, and
a variable communication port and a blowing fan are further provided, the variable communication port being installed in a wall between the bathroom and the patient room and configured to allow communication between the bathroom and the patient room or release communication between the bathroom and the patient room,
the blowing fan being installed in the variable communication port,
wherein negative pressure of the bathroom is generated by the variable communication port and the blowing fan.

5. The negative pressure room of claim 1, wherein the negative pressure device comprises:
a body;
a motor installed inside or outside the body;
an intake part formed at a side of the body, and configured to allow drawing of air in the interior space therethrough;
a filter part provided inside the body, and configured to purify air drawn through the intake part; and
an exhaust part formed on the outside of the body, and configured to allow exhaust of air purified by the filter part therethrough.

6. The negative pressure room of claim 5, wherein the filter part comprises a plurality of filters.

7. The negative pressure room of claim 5, wherein the filter part comprises:
a casing in which a filter is accommodated;
an air sprayer installed at the casing, and configured to generate an air curtain in a vertical direction; and
at least one chemical sprayer installed at the casing at a position between the air curtain and the filter, and configured to spray a chemical toward the filter,
wherein the air curtain prevents the viruses attached to the filter from escaping back in the form of an aerosol due to the chemical sprayer.

8. The negative pressure room of claim 7, wherein the chemical sprayer sprays an aqueous sodium hypochlorite solution.

9. The negative pressure room of claim 1, wherein the first door and the second door are controlled so as not to be opened simultaneously.

10. The negative pressure room of claim 1, further comprising:
a sterilization system installed at a position in the vicinity of the first door of the ante-room, and configured to spray a chemical onto a person entering and leaving the ante-room.
